(19) United States
(12) Patent Application Publication
(10) Pub. No.: US 2005/0038476 A1
(43) Pub. Date: Feb. 17, 2005

(54) Coating/covering materials for the enhancement of defibrillation thresholds of implantable defibrillators/leads

(75) Inventor: Ward M. Brown, La Crosse, WI (US)

Correspondence Address: OPPENHEIMER WOLFF & DONNELLY LLP, 45 SOUTH SEVENTH STREET, SUITE 3300, MINNEAPOLIS, MN, 55402, US

(73) Assignee: Team Brown Enterprises, LLC, La Crosse, WI (US)

(21) Appl. No.: 10/918,165

(22) Filed: Aug. 13, 2004

Related U.S. Application Data
(60) Provisional application No. 60/495,235, filed on Aug. 14, 2003.

(51) Int. Cl. A61N 1/375; A61N 1/05

(52) U.S. Cl. 607/5; 607/36; 607/142

(57) ABSTRACT

The efficiency and longevity of an implantable electrotherapy device is greatly improved by utilizing a biocompatible coating material to selectively cover desired portions of the electrotherapy device and/or the related leads. This coating thus allows only specific portions of the device to be in electrical contact with the patient's tissue, thus more closely controlling the electrical signal transfer characteristics of the device. Because a more concentrated or localized signal transmission characteristic is possible, the efficiency of the electrotherapy device is greatly improved. Further, because power is being more efficiently and effectively utilized, the overall longevity of the implantable device is also improved.

This application claims priority from provisional application Ser. No. 60/495,235, filed Aug. 14, 2003.

### BACKGROUND OF THE INVENTION

The present invention generally relates to implantable electro-stimulation devices for use in the human body. More specifically, the present invention relates to the specific configuration and tailoring of non-conducting coatings so that electrical stimulation signals are more carefully controlled through the remaining surfaces of the device.

The human heart normally maintains its own intrinsic rhythm in order to consistently pump a proper supply of blood throughout the body's circulatory system. However, some people are afflicted with irregular cardiac rhythms, or cardiac arrhythmias, resulting in diminished blood circulation. Drug therapy is one mode of treatment for cardiac arrhythmias. Unfortunately, drug therapy is not effective for treating all cardiac arrhythmias. Hence, alternative modes of treatment including an implantable electrotherapy devices, such as pacemakers and defibrillators, are utilized.

Patients with bradyarrythmias, or symptomatic or slow beating of the heart, are often treated with pacemakers. These devices deliver timed sequences of low energy electrical stimuli to the heart via leads having one or more electrodes placed about the heart. With proper timing of the electrical stimuli, heart contractions are regulated such that the heart contracts at a proper rate, greatly improving blood supply throughout the body's circulatory system.

Patients with malignant tachyarrhythmia, or potentially life threatening fast beating of the heart, are often treated with implantable cardioverter defibrillators. These devices deliver high-energy electrical stimuli called defibrillation countershock to the heart. The countershock interrupts the tachyarrhythmia allowing the heart to establish a perfusing rhythm which allows the heart to completely fill with blood before pumping. Other implantable electrotherapy devices include pacer/defibrillators, which combine the functions of pacemakers and defibrillators, drug delivery devices, and other systems designed for diagnosing and treating arrhythmias.

In addition to the above heart conditions that are treated with electrical stimulation signals, various muscle and nerve conditions also benefit from electrical stimulation. For example, electrical signals may be used for pain management, where signals effect nerve system reaction. Further, electrical systems also include muscular stimulation devices, which provide appropriate-signals to the body to aid in injury recovery. In another example, drug delivery is achieved using electrical signals to “drive” certain drugs into the body.

Conventional implantable electrode leads used together with implantable electrotherapy devices are commonly known. An implantable electrode lead is generally comprised of at least one electrode for supplying an electrical stimulation pulse or sensing an electrically evoked response of the heart, an electrical connector for connecting the electrode lead to an implantable electrotherapy device, and a lead body inserted between the electrode and the electrical connector for transmitting an electrical signal between the electrode and the implantable electrotherapy device. In some cases the connector is eliminated as the lead body is directly connected to the therapy device.

When providing electrical signals to the human body, a minimum threshold signal level is often required in order to establish current flow. Once achieved, the same signal flow can be maintained using less overall power. Further, this threshold becomes a major design consideration for the implantable device. The threshold itself generally relates to the electrical characteristics of human tissue. Further, the configuration of the electrodes themselves will affect the amount of power required to overcome this threshold (which is often measured in terms of current density amperes per centimeters squared). Thus, the surface area of the electrodes will have a direct affect on the threshold current achieved, and thus the overall power required for any particular device.

Biocompatible materials have been developed for covering implantable leads with dissolvable coatings for improving initial placement and extraction of leads and electrodes. In addition these coatings help to minimize infection and scar tissue development around leads and electrodes. (See e.g., U.S. Pat. No. 6,584,363 B2).

In addition to the electrodes, the implantable device itself is a source of electrical current. The configuration of the device's surface area will also effect the electrical operation of the device. Hence, there is currently a need to control the surface area of these electrical components that are exposed to patient tissue when the electrical signals are emitted from the implantable electrotherapy device (including pacemaker or defibrillator surfaces and lead and electrode surfaces).

### BRIEF SUMMARY OF THE INVENTION

Implantable electrotherapy devices (e.g., pacemakers, defibrillators, etc.) are all specifically configured to provide a desired electrical signal to a patient. Often, these devices include control or signal generation circuitry that is included within a “package” or “can”. Further, these devices often utilize electrodes, which are placed some distance away from the main device body. The “can” is also configured to operate as an electrode in the system, thus creating a current path when combined with the remote electrodes.

At the present time, the entire implantable device package is utilized as one electrode. The present invention utilizes a biologically stable, non-conductive coating material applied to the device package to modify the amount of surface area exposed to human tissue. Additionally, the electrodes cooperating with the device package are similarly coated in order to provide only a limited amount of exposed area. By eliminating the conducting surface area exposed to patient tissue, the current density may be more easily controlled. More specifically, the same amount of current density can be achieved using much lower current levels. Because lower current levels would be required, the efficiency of the overall system is enhanced. Additionally, the longevity of the implantable electrotherapy device is increased because lower power levels are required.

### BRIEF DESCRIPTION OF THE DRAWINGS

Further details of the preferred embodiment can be seen by reading the following description in conjunction with the drawings in which:

FIG. 1 is a perspective view of an embodiment of an implantable electrotherapy device of the present invention;

FIG. 2 is a side elevation of an embodiment of an implantable electrotherapy device of the present invention;

FIG. 3 is a bottom plan view of an embodiment of a implantable electrotherapy device of the present invention;

FIG. 4 is a perspective view of a template of the present invention; and,

FIG. 5 is a top view of lead for use in an implantable electrotherapy system.

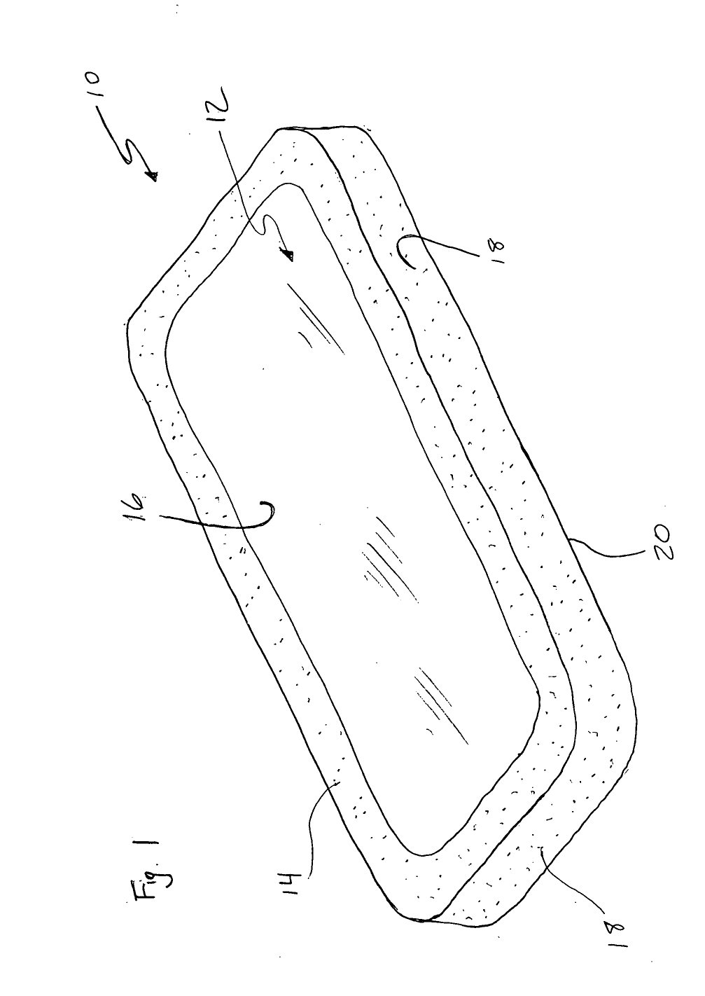

### DETAILED DESCRIPTION OF THE INVENTION

The present invention pertains to a non-conductive, biocompatible coating, such as a polymer, ceramic, Teflon or silicon based material for use in conjunction with an implantable electrotherapy device. More specifically, it pertains to the use of such a coating on specific surfaces of the implantable electrotherapy device and leads in order to selectively enhance and control of the delivery of electrical current to the surrounding human tissue.

Referring to the drawings, a device 10 of the present invention is depicted. Preferably, the device 10 comprises an implantable electrotherapy device 10. It will be understood that electrotherapy device 10 contains electrical circuitry required to generate all electrotherapy signals desired. The bottom surface 12 of the implantable electrotherapy device may be coated with a non-conductive, biocompatible material 14 such that some portion 16 of the surface remains uncoated and electrically active. The remaining surfaces of the implantable electrotherapy device, including the four sides 18 and top 20, may also be coated with a non-conductive, biocompatible material.

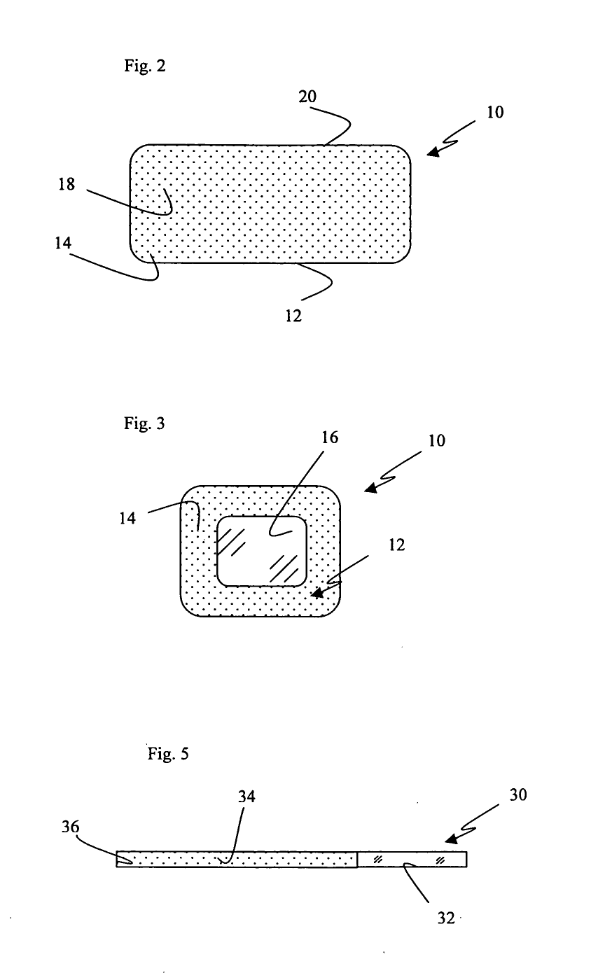

FIG. 2 depicts a side view of the implantable electrotherapy device 10 where the entire side 18 is coated with a non-conductive, biocompatible material 14 from the bottom 12 to the top 20 of the device. All six sides of the implantable electrotherapy device 10 may be coated in a similar fashion with a non-conductive, biocompatible material. FIG. 3 again depicts the bottom surface 20 of the implantable electrotherapy device where some portion of the bottom surface is coated with a non-conductive, biocompatible material 14 while an alternative portion 16 of the bottom surface 12 remains uncoated and electrically active.

The portion 16 of the implantable electrotherapy device that remains uncoated will allow electric current from the device to enter the surrounding human tissue in a specific, localized fashion. In this manner, a predetermined current density may be delivered to a specific area in the body. Allowing this specificity for the delivery of the electrical current will improve the performance of the implantable electrotherapy device for stimulating heart response. In addition, the implantable electrotherapy device 10 will operate more precisely for a longer period of time because less energy will be used.

The size of the exposed area 16 is proportional to the current density that passes therethrough. Thus, one aspect of the present invention provides a coating 14 that can be removed from the device by a physician or technician in order to custom-fit the size of the exposed area to the application. FIG. 3 shows an embodiment of the present invention that includes a device 10 that is completely covered with a non-conductive biocompatible material 14. One or more rings 22 are drawn on the covering 14 on one of the surfaces of the device 10. These rings 22 are guidelines to be used when removing the material 14 to form uncovered portions 16 of various sizes. It is envisioned that removal may be accomplished by cutting along the rings 22 with a blade and peeling away the material 14 within the ring. Alternatively, the material may comprise a coating, rather than a covering, that may be scraped away from the surface of the device 10. Furthermore, as depicted in FIG. 4, a template 24 may be provided with cutouts 26 of various sizes to assist the physician or technician in cutting or scraping away a desired amount of material 14. The template 24 may be used with or without the rings 22.

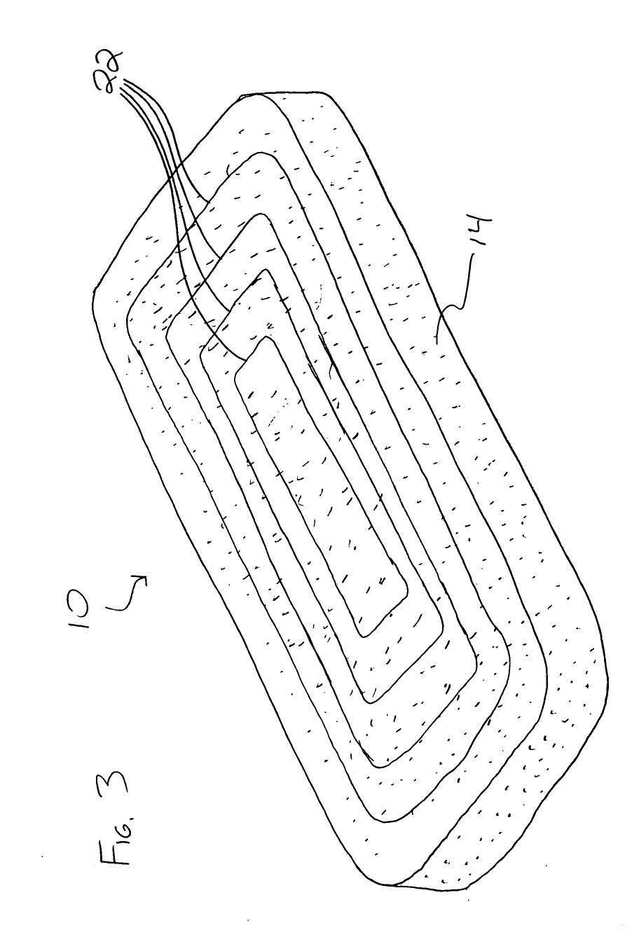

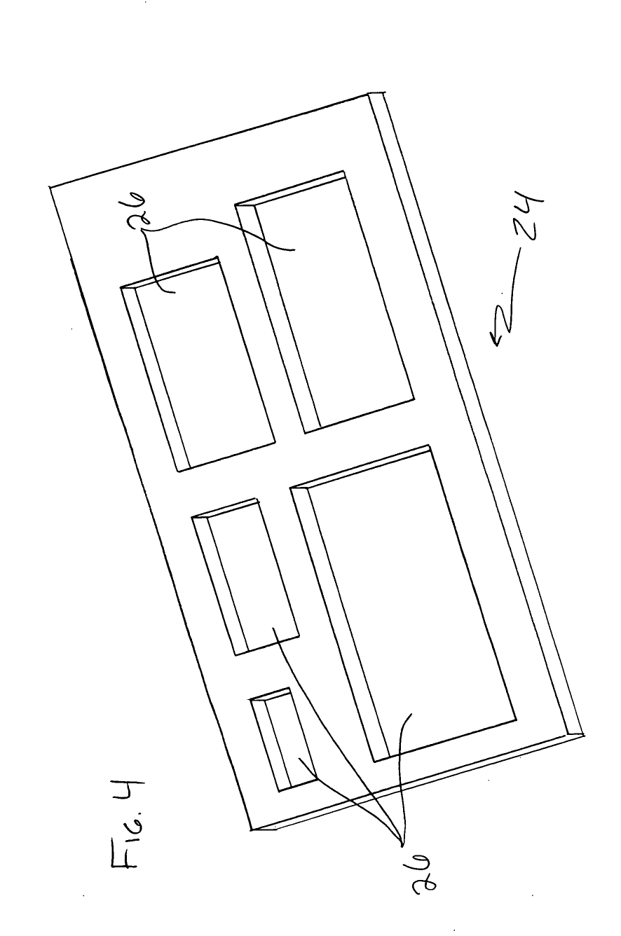

Referring now to FIG. 5, a lead 30 is shown for use with an implantable electrotherapy device. Some portion of the lead 30 may be coated with a non-conductive, biocompatible material 34 while the remaining portion 32 of the lead may remain uncoated. The uncoated portion 32 of the lead 30 will be electrically active. In the same manner as the uncoated 16 portion of the implantable electrotherapy device, by specifying the portion 32 of the lead that will remain uncoated, the current density delivered by the implantable electrotherapy device may be controlled. The results will be improved performance of the implantable electrotherapy device for maintaining proper cardiac rhythm (or other desired functions), and more efficient energy consumption and increased longevity of the implantable electrotherapy device. A desired length of material 34 may be stripped from the lead 30 in the same manner that insulation is stripped from a wire.

The foregoing detailed description of the preferred embodiments of the invention has been provided for the purposes of illustration and description. It is not intended to be exhaustive or to limit the invention to the precise embodiments disclosed. Many modifications and variations will be apparent to practitioners skilled in this art. The embodiments were chosen and described in order to best explain the principles of the invention and its practical application, thereby enabling others skilled in the art to understand the invention for various embodiments and with various modifications as are suited to the particular use contemplated. It is intended that the scope of the invention be defined by the following claims and their equivalents.

1. An implantable electrotherapy device for providing electrical signals to a patient, comprising:
a signal generation device contained within a device container, the device container primarily constructed of electrically conductive material;
an electrode electrically connected to the signal generation device, the electrode and signal generation device cooperating with one another to provide electrotherapy signals to the patient; and
a biocompatible coating selectively applied to predetermined portions of the device container or the electrode so as to provide tailored generation of the therapy signals to predetermined portions of the patient's tissue.

2. The device of claim 1 wherein the device container is rectangular in shape, and all surfaces except one are completely coated with the biocompatible coating.

3. The device of claim 1 wherein the coating is a ceramic material.

4. The device of claim 1 wherein the coating is a silicon based material.

5. The device of claim 1 wherein the coating is a polymer material.

6. The device of claim 1 wherein the coating is Teflon.

7. The device of claim 1 wherein the biocompatible coating is applied to predetermined portions of both the device container and the electrode so as to provide tailored generation of the therapy signals to predetermined portions of the patient's tissue.

8. The device of claim 1 wherein the biocompatible coating is applied only to predetermined portions of the device container.

9. The device of claim 1 wherein the biocompatible coating is constructed and arranged such that a portion of the coating may be removed subsequent to production.

10. A method of controlling current density generated by an implantable electrotherapy device having a conductive container acting as an electrode comprising:
identifying an area of a predetermined size on the container to act as an electrode providing a desired current density;
electrically insulating outside surfaces of the container with the exception of the identified area.

11. The method of claim 10 wherein electrically insulating outside surfaces of the container with the exception of the identified area comprises coating the outside surfaces with an electrically insulating material with the exception of the identified area.

12. The method of claim 11 wherein coating the outside surfaces with an electrically insulating material with the exception of the identified area comprises coating the outside surfaces with a material belonging to the group consisting of polymer, ceramic, Teflon, and silicon-based material.

13. The method of claim 10 wherein electrically insulating outside surfaces of the container with the exception of the identified area comprises covering the outside surfaces with an electrically insulating material with the exception of the identified area.

14. The method of claim 13 wherein covering the outside surfaces with an electrically insulating material with the exception of the identified area comprises covering the outside surfaces with a material belonging to the group consisting of polymer, ceramic, Teflon, and silicon-based material.

15. The method of claim 10 wherein electrically insulating outside surfaces of the container with the exception of the identified area comprises removing insulating material from the identified area and leaving insulating material outside of the identified area intact.

16. An implantable device useable to transmit electrical signals to a patient comprising:
electrical circuitry capable of generating electrotherapy signals;
an electrically conductive housing surrounding the electrical circuitry, the housing operably connected thereto such that the electrically conductive housing may act as an electrode;
electrically insulative material surrounding selected outer surfaces of the housing, thereby limiting areas of the housing that may act as an electrode;
at least one external electrode remote from the housing and electrically connected to the electrical circuitry via a lead passing through the housing and electrically insulated therefrom.

17. The implantable device of claim 16 wherein the electrically conductive housing comprises a plurality of faces, all but one of the faces surrounded by electrically insulative material.

18. The implantable device of claim 16 wherein the electrically insulative material comprises a biocompatible coating.

19. The implantable device of claim 16 wherein the electrically insulative material comprises a biocompatible covering.

20. The implantable device of claim 16 wherein the electrically conductive housing comprises a plurality of faces, all but one of the faces completely insulated by the insulative material, the one face not completely insulated by the insulative material being partially insulated by the insulative material.

21. The implantable device of claim 16 wherein the insulative material belongs to the group consisting of material belonging to the group consisting of polymer, ceramic, Teflon, and silicon-based material.

22. The implantable device of claim 16 wherein the at least one external electrode remote from the housing is partially surrounded by the electrically insulative material.
